(19) United States
(12) Patent Application Publication
(10) Pub. No.: US 2024/0410006 A1
(43) Pub. Date: Dec. 12, 2024

(54) Method for Predicting Health of a Human Subject

(71) Applicant: Tru Diagnostics, Inc., Lexington, KY (US)

(72) Inventors: Varun Dwaraka, Lexington, KY (US); Hannah Went, Nicholasville, KY (US); Ryan Smith, Nicholasville, KY (US)

(21) Appl. No.: 18/741,311

(22) Filed: Jun. 12, 2024

Related U.S. Application Data
(60) Provisional application No. 63/472,428, filed on Jun. 12, 2023.

(51) Int. Cl. C12Q 1/6883; C12Q 1/6837; C12Q 1/686; G16H 50/30

(52) U.S. Cl. CPC C12Q 1/6883; C12Q 1/6837; C12Q 1/686; G16H 50/30; C12Q 2600/154

(57) ABSTRACT

A method for predicting the health of a human subject includes steps of: (a) obtaining genomic DNA from a blood or saliva specimen of a human subject, (b) observing unmethylated CG loci, wherein said observing includes performing a bisulfite conversion process on the genomic DNA of the human subject, (c) comparing the unmethylated CG loci observed in (b) to unmethylated CG loci observed in genomic DNA collected from a reference group of human individuals, and (d) correlating the unmethylated CG loci observed in (b) with the unmethylated CG loci observed in the reference group of human individuals to predict health of the human subject.

### RELATED APPLICATION

This patent application claims priority to U.S. Provisional Patent Application Ser. No. 63/472,428, filed on Jun. 12, 2023, the full disclosure of which is incorporated herein by reference.

### TECHNICAL FIELD

This document generally relates to the construction and validation of a DNA reference matrix focusing on certain unmethylated cytosine-phosphate-guanine sites/CG loci in order to (a) predict levels of a plurality of proteins and metabolites in a human subject to be tested and (b) correlate the predicted levels of the plurality of proteins and metabolites with disease risk for the human subject. In this way it is possible to better correlate patterns to disease and to create more complex phenotype (disease) prediction with more information through methylation.

### BACKGROUND

When your body is aging faster than the calendar's progression of time, you are experiencing accelerated biological aging; the #1 predictor of chronic disease. Fortunately, there are lifestyle and medical interventions proven to help slow, stop, or even reverse the biological aging processes in your body. This document relates to a method for measuring baseline and changes to an individual's pace of aging, giving that individual the power to steer his or her anti-aging or health and wellness journey in the right direction.

### SUMMARY

A new and improved method is provided for predicting the health of a human subject. That method comprises, consists of or consists essentially of the steps of:
(a) obtaining genomic DNA from a blood or saliva specimen of the human subject in a manner known in the art;
(b) observing unmethylated CG loci, in the genomic DNA of the human subject, wherein said observing includes performing a bisulfite conversion process on the genomic DNA of the human subject so that cytosine residues in the genomic DNA of the human subject are transformed to uracil, while 5-methylcytosine residues in the genomic DNA of the human subject are not transformed to uracil;
(c) comparing the unmethylated CG loci observed in (b) to unmethylated CG loci observed in genomic DNA collected from a reference group of human individuals; and
(d) correlating the unmethylated CG loci observed in (b) with the unmethylated CG loci observed in the reference group of human individuals to predict health of the human subject wherein the CG loci are a plurality of CG loci selected from a group consisting of: cg00011943, cg00014020, cg00056433, cg00067133, cg00071360, cg00074145, cg00080105, cg00120123, cg00122614, cg00153759, cg00177243, cg00178119, cg00182994, cg00209129, cg00215550, cg00217225, cg00236988, cg00237714, cg00243480, cg00251125, cg00262132, cg00275686, cg00278547, cg00283022, cg00303773, cg00322993, cg00342468, cg00358895, cg00458337, cg00465312, cg00481382, cg00530438, cg00538458, cg00548856, cg00593298, cg00594167, cg00602326, cg00615271, cg00631329, cg00639633, cg00646555, cg00687674, cg00687962, cg00698355, cg00712841, cg00737155, cg00748449, cg00756943, cg00767058, cg00800095, cg00815399, cg00910115, cg00935026, cg00935421, cg00964035, cg00973876, cg00993310, cg01020263, cg01025883, cg01067216, cg01089474, cg01094440, cg01098936, cg01113710, cg01134012, cg01166985, cg01169463, cg01178063, cg01224520, cg01243823, cg01253289, cg01266159, cg01271129, cg01274656, cg01282852, cg01313513, cg01320510, cg01346718, cg01377932, cg01380346, cg01402735, cg01410359, cg01446515, cg01457077, cg01541424, cg01545493, cg01549940, cg01579765, cg01586635, cg01606057, cg01610915, cg01645869, cg01652244, cg01660630, cg01715525, cg01770232, cg01809675, cg01824466, cg01826979, cg01844642, cg01849284, cg01857379, cg01858517, cg01865937, cg01892689, cg01893681, cg01894498, cg01929138, cg01930417, cg01956293, cg02001279, cg02007867, cg02042600, cg02048674, cg02055264, cg02056921, cg02058108, cg02081263, cg02084118, cg02084211, cg02094681, cg02106682, cg02132714, cg02217269, cg02246605, cg02310386, cg02339455, cg02345572, cg02371935, cg02380750, cg02390624, cg02415029, cg02426324, cg02444928, cg02447542, cg02471378, cg02474731, cg02489377, cg02500990, cg02509300, cg02524236, cg02534780, cg02605776, cg02613370, cg02618485, cg02643782, cg02672830, cg02675560, cg02720346, cg02721394, cg02731293, cg02740457, cg02762363, cg02768694, cg02798801, cg02867402, cg02909068, cg02942382, cg02981003, cg02987388, cg03007462, cg03031520, cg03044367, cg03082247, cg03137472, cg03138091, cg03139710, cg03240800, cg03245912, cg03261737, cg03328727, cg03340356, cg03340754, cg03356492, cg03357219, cg03368099, cg03400443, cg03401077, cg03417454, cg03423767, cg03441242, cg03443888, cg03458265, cg03463465, cg03474430, cg03514545, cg03517760, cg03546163, cg03549506, cg03549779, cg03550384, cg03558379, cg03587144, cg03593336, cg03605735, cg03626541, cg03636183, cg03644691, cg03650179, cg03654623, cg03676636, cg03707183, cg03732728, cg03741185, cg03741498, cg03743982, cg03760060, cg03772253, cg03785619, cg03796580, cg03807316, cg03834947, cg03839794, cg03858365, cg03874914, cg03876766, cg03877420, cg03926921, cg03935495, cg03945604, cg03989967, cg04030486, cg04045009, cg04046848, cg04060356, cg04064054, cg04079779, cg04083076, cg04114590, cg04115418, cg04137003, cg04139465, cg04148839, cg04156293, cg04161236, cg04162034, cg04172953, cg04181107, cg04204356, cg04206342, cg04213565, cg04214966, cg04225172, cg04227949, cg04230438, cg04235071, cg04241623, cg04276953, cg04285652, cg04322572, cg04393397, cg04398451, cg04411086, cg04436120, cg04439218, cg04444415, cg04460372, cg04480106, cg04484376, cg04545003, cg04567124, cg04584675, cg04595658, cg04615668, cg04707706, cg04733838, cg04742282, cg04757168, cg04768425, cg04781339, cg04789125, cg04799410, cg04836038, cg04846343, cg04847826, cg04859706, cg04875128, cg04885881, cg04902443, cg04907325, cg04980928, cg05021355, cg05024939, cg05067995, cg05169665, cg05185749, cg05187965, cg05196720, cg05201093, cg05230392, cg05236757, cg05239544, cg05258736, cg05270922, cg05280814, cg05298224, cg05331789, cg05338009, cg05352464, cg05380920, cg05399221, cg05418947, cg05422344, cg05423392, cg05426134, cg05442477, cg05481257, cg05485520, cg05498943, cg05506600, cg05517880, cg05551469, cg05575921, cg05616010, cg05635388, cg05666515, cg05669298, cg05671603, cg05676441, cg05679108, cg05715050, cg05720678, cg05721485, cg05726118, cg05729185, cg05777089, cg05786053, cg05802075, cg05820243, cg05828191, cg05830220, cg05845765, cg05901141, cg05910038, cg05913921, cg05941525, cg05951558, cg05987787, cg05989533, cg06001484, cg06030695, cg06047828, cg06062604, cg06068545, cg06072257, cg06083423, cg06159131, cg06169746, cg06213468, cg06232874, cg06251791, cg06279632, cg06295952, cg06319999, cg06335143, cg06374715, cg06401979, cg06429010, cg06454296, cg06478823, cg06519334, cg06535156, cg06572713, cg06572849, cg06580943, cg06602086, cg06604227, cg06610988, cg06621358, cg06630425, cg06639320, cg06642354, cg06645033, cg06660395, cg06665890, cg06667732, cg06672424, cg06675893, cg06679261, cg06683719, cg06692384, cg06696596, cg06753303, cg06784991, cg06789517, cg06812822, cg06870213, cg06874063, cg06878994, cg06888566, cg06926961, cg06958563, cg06975196, cg06984463, cg06997226, cg07015424, cg07023764, cg07057549, cg07076816, cg07097098, cg07151966, cg07193470, cg07196593, cg07202981, cg07304986, cg07323488, cg07351787, cg07363137, cg07365979, cg07393369, cg07404706, cg07426101, cg07434188, cg07462861, cg07529122, cg07541744, cg07558704, cg07569063, cg07601816, cg07613945, cg07620230, cg07624842, cg07629158, cg07640648, cg07643355, cg07668501, cg07672814, cg07674095, cg07675998, cg07692435, cg07702236, cg07714289, cg07768696, cg07778205, cg07828121, cg07855914, cg07871442, cg07871717, cg07899016, cg07951728, cg08075719, cg08094551, cg08103988, cg08113373, cg08125436, cg08126148, cg08132054, cg08173263, cg08182270, cg08259413, cg08281455, cg08284801, cg08312389, cg08335107, cg08342619, cg08350092, cg08369164, cg08375139, cg08421807, cg08424491, cg08439752, cg08443038, cg08506743, cg08551532, cg08573404, cg08609249, cg08640901, cg08684580, cg08709765, cg08743947, cg08750459, cg08762603, cg08772194, cg08812936, cg08830214, cg08856764, cg08885224, cg08893036, cg08968830, cg08992229, cg09082985, cg09087222, cg09087669, cg09094866, cg09103812, cg09129997, cg09146197, cg09167379, cg09169335, cg09191731, cg09196193, cg09211372, cg09228052, cg09267188, cg09313734, cg09317728, cg09365138, cg09391799, cg09422291, cg09428537, cg09434029, cg09435875, cg09450907, cg09463001, cg09507782, cg09536390, cg09554406, cg09576223, cg09601629, cg09621460, cg09681594, cg09702859, cg09703048, cg09716613, cg09717927, cg09717936, cg09788524, cg09823288, cg09823713, cg09831942, cg09842685, cg09862733, cg09868598, cg09874992, cg09931939, cg09963257, cg09969145, cg09977126, cg09993780, cg10043414, cg10060499, cg10116893, cg10123377, cg10127085, cg10133386, cg10157184, cg10168894, cg10201382, cg10208550, cg10235817, cg10251070, cg10290474, cg10325507, cg10326287, cg10407585, cg10459672, cg10472537, cg10501210, cg10514580, cg10518439, cg10573018, cg10583266, cg10601230, cg10621420, cg10624914, cg10662975, cg10703784, cg10716444, cg10759025, cg10766585, cg10859114, cg10866445, cg10874644, cg10878114, cg10882522, cg10884112, cg10949164, cg10961323, cg10977392, cg10996589, cg11020001, cg11031278, cg11052535, cg11104177, cg11113469, cg11120021, cg11130780, cg11149163, cg11240908, cg11241278, cg11270070, cg11290628, cg11338041, cg11341981, cg11362848, cg11400286, cg11408477, cg11409205, cg11415498, cg11426563, cg11479653, cg11532431, cg11540692, cg11620909, cg11624211, cg11640253, cg11649376, cg11671400, cg11691908, cg11725835, cg11735780, cg11737758, cg11749817, cg11752250, cg11765003, cg11777043, cg11793132, cg11801086, cg11804239, cg11819402, cg11824202, cg11833003, cg11844835, cg11921111, cg11935615, cg11951427, cg11971423, cg11976052, cg12016314, cg12016809, cg12018436, cg12030941, cg12038299, cg12050358, cg12100785, cg12112853, cg12135976, cg12145721, cg12146151, cg12151545, cg12158389, cg12159504, cg12168623, cg12170419, cg12195980, cg12242038, cg12272488, cg12282371, cg12307092, cg12347617, cg12364808, cg12378187, cg12394821, cg12416359, cg12434362, cg12463857, cg12518665, cg12538421, cg12560256, cg12569593, cg12586992, cg12606517, cg12615235, cg12637448, cg12655260, cg12662084, cg12743638, cg12744031, cg12797254, cg12853426, cg12935715, cg12941754, cg12978573, cg12986327, cg13017801, cg13055484, cg13073141, cg13083251, cg13087259, cg13099106, cg13154925, cg13167828, cg13169967, cg13176778, cg13188098, cg13206721, cg13224583, cg13229291, cg13234653, cg13257436, cg13295463, cg13298859, cg13312094, cg13340220, cg13343932, cg13367272, cg13369416, cg13372562, cg13393601, cg13399544, cg13431929, cg13473880, cg13507563, cg13543436, cg13545297, cg13563074, cg13609948, cg13671730, cg13672444, cg13676215, cg13690812, cg13697829, cg13702196, cg13714975, cg13719443, cg13742305, cg13779009, cg13814747, cg13897675, cg13914486, cg13941682, cg13959647, cg13979305, cg13987110, cg14030785, cg14047961, cg14117377, cg14118991, cg14157578, cg14167430, cg14172633, cg14175748, cg14209041, cg14224028, cg14234520, cg14251509, cg14252140, cg14257435, cg14262357, cg14264125, cg14280181, cg14339287, cg14354398, cg14384748, cg14391737, cg14413904, cg14441271, cg14442890, cg14484844, cg14534474, cg14615491, cg14639293, cg14640588, cg14675598, cg14688662, cg14708893, cg14710896, cg14738170, cg14751023, cg14781394, cg14794281, cg14801398, cg14837598, cg14850945, cg14873958, cg14920334, cg14942542, cg14962363, cg14975009, cg14994914, cg15033140, cg15047582, cg15059204, cg15109292, cg15128785, cg15141757, cg15143202, cg15175143, cg15179011, cg15194100, cg15208687, cg15223345, cg15234627, cg15246131, cg15288815, cg15318748, cg15374686, cg15391651, cg15405515, cg15418593, cg15436354, cg15467112, cg15471501, cg15480367, cg15498435, cg15506060, cg15575700, cg15592143, cg15603964, cg15625940, cg15690995, cg15709989, cg15710198, cg15720821, cg15733713, cg15821589, cg15827092, cg15837235, cg15885703, cg15909933, cg15961042, cg15968648, cg15994304, cg16014202, cg16014412, cg16026114, cg16070035, cg16084969, cg16097191, cg16141165, cg16195970, cg16198075, cg16211224, cg16240781, cg16306900, cg16314089, cg16369168, cg16408865, cg16430267, cg16488565, cg16489878, cg16517790, cg16529483, cg16551483, cg16569522, cg16570202, cg16649588, cg16661213, cg16679001, cg16733203, cg16736896, cg16769369, cg16795184, cg16842060, cg16861968, cg16882373, cg16891431, cg16908208, cg16969368, cg16979095, cg16987876, cg16994082, cg17017746, cg17030092, cg17050472, cg17077421, cg17107501, cg17117243, cg17120358, cg17129809, cg17133012, cg17148219, cg17210497, cg17219432, cg17225923, cg17232357, cg17251114, cg17275047, cg17408993, cg17435901, cg17464298, cg17464436, cg17465569, cg17476910, cg17485717, cg17514701, cg17569235, cg17576228, cg17591595, cg17652174, cg17660663, cg17665268, cg17691612, cg17695543, cg17699330, cg17761940, cg17786697, cg17825846, cg17872672, cg17885794, cg17944885, cg17945419, cg17967443, cg17979595, cg17981101, cg17988830, cg18014413, cg18023598, cg18031708, cg18031880, cg18071894, cg18133309, cg18181703, cg18190506, cg18251449, cg18262201, cg18319024, cg18380497, cg18398019, cg18425341, cg18516816, cg18587063, cg18597252, cg18598029, cg18604560, cg18669861, cg18699242, cg18740583, cg18771034, cg18784500, cg18795169, cg18817318, cg18822243, cg18856359, cg18859711, cg18882407, cg18885210, cg18976649, cg19006431, cg19009870, cg19058685, cg19089073, cg19091784, cg19172278, cg19182279, cg19224645, cg19225755, cg19266329, cg19284211, cg19296509, cg19297245, cg19302996, cg19304797, cg19308663, cg19320294, cg19380156, cg19474865, cg19536401, cg19548511, cg19558933, cg19604384, cg19611570, cg19635897, cg19636673, cg19652271, cg19665696, cg19693031, cg19726712, cg19730248, cg19774733, cg19800670, cg19822434, cg19859910, cg19909400, cg19935065, cg19941446, cg19977924, cg19992304, cg20004147, cg20025086, cg20037601, cg20059597, cg20119148, cg20128319, cg20143092, cg20155714, cg20162239, cg20185773, cg20192259, cg20207443, cg20217702, cg20244340, cg20249402, cg20270599, cg20272789, cg20282550, cg20299697, cg20348196, cg20351734, cg20353489, cg20370296, cg20376953, cg20432211, cg20442128, cg20446824, cg20515136, cg20534585, cg20587151, cg20594802, cg20596493, cg20597382, cg20597477, cg20601028, cg20623853, cg20628696, cg20656525, cg20663241, cg20668177, cg20675702, cg20720208, cg20737582, cg20773320, cg20802942, cg20865054, cg20874261, cg20891771, cg20907051, cg20914958, cg20918218, cg20921972, cg20972814, cg20973476, cg21000454, cg21077074, cg21082913, cg21112943, cg21123355, cg21124655, cg21132686, cg21136789, cg21185661, cg21204522, cg21232671, cg21253043, cg21259215, cg21305555, cg21306240, cg21312434, cg21314942, cg21321148, cg21346818, cg21356998, cg21410629, cg21447975, cg21455901, cg21489638, cg21533743, cg21566642, cg21622536, cg21628390, cg21640508, cg21658235, cg21693883, cg21717369, cg21723004, cg21725954, cg21730080, cg21733098, cg21739934, cg21761881, cg21783047, cg21791657, cg21824343, cg21834645, cg21870393, cg21907521, cg21934504, cg21947254, cg21962907, cg21985831, cg21986679, cg21998324, cg22002034, cg22019884, cg22022821, cg22023206, cg22041417, cg22061523, cg22061769, cg22069247, cg22171714, cg22178367, cg22200330, cg22220710, cg22232492, cg22243260, cg22282672, cg22322653, cg22339775, cg22344631, cg22356226, cg22363327, cg22416596, cg22434421, cg22442430, cg22454769, cg22556008, cg22563987, cg22581353, cg22594985, cg22691256, cg22711187, cg22719651, cg22729726, cg22736354, cg22792461, cg22822140, cg22822803, cg22945467, cg22956922, cg22993154, cg23059366, cg23063236, cg23080422, cg23192918, cg23196035, cg23234847, cg23235141, cg23248055, cg23265105, cg23276290, cg23302570, cg23313424, cg23366493, cg23371851, cg23378546, cg23410824, cg23424407, cg23447131, cg23465508, cg23486701, cg23500537, cg23547619, cg23549902, cg23561053, cg23570968, cg23574427, cg23586821, cg23615892, cg23643435, cg23793194, cg23838503, cg23879118, cg23883522, cg23894058, cg23910413, cg23923562, cg23956385, cg23964820, cg24003560, cg24027965, cg24055030, cg24055461, cg24112628, cg24141036, cg24185013, cg24220948, cg24236938, cg24241488, cg24262996, cg24266506, cg24293344, cg24317857, cg24337318, cg24354818, cg24362016, cg24365657, cg24372256, cg24392971, cg24397183, cg24432675, cg24463073, cg24468603, cg24492140, cg24507955, cg24520070, cg24523415, cg24530234, cg24543255, cg24577941, cg24589102, cg24601404, cg24674445, cg24678611, cg24697099, cg24721873, cg24769628, cg24796651, cg24799795, cg24835295, cg24920358, cg24925701, cg24967099, cg24976744, cg24982343, cg24997750, cg25047618, cg25063538, cg25114611, cg25146575, cg25148589, cg25156198, cg25161899, cg25181997, cg25189904, cg25202593, cg25259149, cg25259707, cg25330725, cg25331593, cg25361850, cg25375711, cg25397922, cg25418001, cg25418670, cg25420482, cg25420747, cg25436217, cg25499552, cg25504214, cg25560772, cg25563198, cg25569590, cg25623035, cg25638549, cg25639566, cg25670171, cg25671708, cg25703588, cg25717260, cg25738786, cg25745729, cg25784308, cg25786980, cg25843115, cg25843873, cg25903319, cg25916759, cg25927838, cg25928819, cg25943804, cg25945293, cg25952581, cg25952596, cg26029902, cg26044490, cg26071551, cg26093148, cg26116542, cg26116556, cg26156626, cg26229024, cg26239485, cg26267310, cg26276120, cg26301353, cg26314853, cg26315261, cg26441486, cg26442630, cg26443646, cg26479667, cg26499822, cg26500093, cg26501477, cg26528000, cg26544857, cg26577320, cg26599630, cg26606789, cg26676765, cg26691434, cg26702770, cg26760942, cg26764761, cg26767387, cg26805839, cg26818257, cg26839512, cg26883048, cg26962439, cg26992381, cg27021634, cg27066201, cg27079776, cg27127090, cg27128734, cg27130665, cg27131142, cg27152890, cg27154679, cg27154725, cg27208536, cg27229100, cg27245649, cg27256066, cg27296119, cg27315314, cg27336261, cg27348423, cg27354537, cg27357902, cg27368039, cg27374247, cg27395675, cg27449200, cg27454650, cg27461310, cg27488658, cg27498980, cg27500647, cg27553457, cg27589058, cg27632248.

The number of CG loci observed from the indicated group of CG loci may vary from embodiment to embodiment of the method. Thus, the plurality of CG loci may be any number between 2 CG loci and 1,352 (all) of the CG loci of the indicated group.

The method may include amplifying the genomic DNA of the blood or saliva specimen by a polymerase chain reaction.

The observing of the unmethylated CG loci, in the genomic DNA of the human subject may include hybridizing the genomic DNA to a beadchip to create a genomic DNA hybridized beadchip and then imaging the genomic DNA hybridized beadchip.

The method may include using a regression algorithm to correlate the unmethylated CG loci observed in (b) with the unmethylated GC loci observed in the reference group of human individuals.

The method may include performing, by computing device, a penalized regression model to create the regression algorithm of CpGs with relative weights wherein a methylation risk score (MRS) is defined by linear combination of m CpG site beta values c and weights w where: MRS=Σi=1mwici.

### DETAILED DESCRIPTION

A method for predicting the health of a human subject includes the steps of:
(a) obtaining genomic DNA from a blood or saliva specimen of the human subject;
(b) observing unmethylated CG loci, in the genomic DNA of the human subject, wherein said observing includes performing a bisulfite conversion process on the genomic DNA of the human subject so that cytosine residues in the genomic DNA of the human subject are transformed to uracil, while 5-methylcytosine residues in the genomic DNA of the human subject are not transformed to uracil;
(c) comparing the unmethylated CG loci observed in (b) to unmethylated CG loci observed in genomic DNA collected from a reference group of human individuals; and
(d) correlating the unmethylated CG loci observed in (b) with the unmethylated CG loci observed in the reference group of human individuals to predict health of the human subject wherein the CG loci are a plurality of CG loci selected from a group consisting of: cg00011943, cg00014020, cg00056433, cg00067133, cg00071360, cg00074145, cg00080105, cg00120123, cg00122614, cg00153759, cg00177243, cg00178119, cg00182994, cg00209129, cg00215550, cg00217225, cg00236988, cg00237714, cg00243480, cg00251125, cg00262132, cg00275686, cg00278547, cg00283022, cg00303773, cg00322993, cg00342468, cg00358895, cg00458337, cg00465312, cg00481382, cg00530438, cg00538458, cg00548856, cg00593298, cg00594167, cg00602326, cg00615271, cg00631329, cg00639633, cg00646555, cg00687674, cg00687962, cg00698355, cg00712841, cg00737155, cg00748449, cg00756943, cg00767058, cg00800095, cg00815399, cg00910115, cg00935026, cg00935421, cg00964035, cg00973876, cg00993310, cg01020263, cg01025883, cg01067216, cg01089474, cg01094440, cg01098936, cg01113710, cg01134012, cg01166985, cg01169463, cg01178063, cg01224520, cg01243823, cg01253289, cg01266159, cg01271129, cg01274656, cg01282852, cg01313513, cg01320510, cg01346718, cg01377932, cg01380346, cg01402735, cg01410359, cg01446515, cg01457077, cg01541424, cg01545493, cg01549940, cg01579765, cg01586635, cg01606057, cg01610915, cg01645869, cg01652244, cg01660630, cg01715525, cg01770232, cg01809675, cg01824466, cg01826979, cg01844642, cg01849284, cg01857379, cg01858517, cg01865937, cg01892689, cg01893681, cg01894498, cg01929138, cg01930417, cg01956293, cg02001279, cg02007867, cg02042600, cg02048674, cg02055264, cg02056921, cg02058108, cg02081263, cg02084118, cg02084211, cg02094681, cg02106682, cg02132714, cg02217269, cg02246605, cg02310386, cg02339455, cg02345572, cg02371935, cg02380750, cg02390624, cg02415029, cg02426324, cg02444928, cg02447542, cg02471378, cg02474731, cg02489377, cg02500990, cg02509300, cg02524236, cg02534780, cg02605776, cg02613370, cg02618485, cg02643782, cg02672830, cg02675560, cg02720346, cg02721394, cg02731293, cg02740457, cg02762363, cg02768694, cg02798801, cg02867402, cg02909068, cg02942382, cg02981003, cg02987388, cg03007462, cg03031520, cg03044367, cg03082247, cg03137472, cg03138091, cg03139710, cg03240800, cg03245912, cg03261737, cg03328727, cg03340356, cg03340754, cg03356492, cg03357219, cg03368099, cg03400443, cg03401077, cg03417454, cg03423767, cg03441242, cg03443888, cg03458265, cg03463465, cg03474430, cg03514545, cg03517760, cg03546163, cg03549506, cg03549779, cg03550384, cg03558379, cg03587144, cg03593336, cg03605735, cg03626541, cg03636183, cg03644691, cg03650179, cg03654623, cg03676636, cg03707183, cg03732728, cg03741185, cg03741498, cg03743982, cg03760060, cg03772253, cg03785619, cg03796580, cg03807316, cg03834947, cg03839794, cg03858365, cg03874914, cg03876766, cg03877420, cg03926921, cg03935495, cg03945604, cg03989967, cg04030486, cg04045009, cg04046848, cg04060356, cg04064054, cg04079779, cg04083076, cg04114590, cg04115418, cg04137003, cg04139465, cg04148839, cg04156293, cg04161236, cg04162034, cg04172953, cg04181107, cg04204356, cg04206342, cg04213565, cg04214966, cg04225172, cg04227949, cg04230438, cg04235071, cg04241623, cg04276953, cg04285652, cg04322572, cg04393397, cg04398451, cg04411086, cg04436120, cg04439218, cg04444415, cg04460372, cg04480106, cg04484376, cg04545003, cg04567124, cg04584675, cg04595658, cg04615668, cg04707706, cg04733838, cg04742282, cg04757168, cg04768425, cg04781339, cg04789125, cg04799410, cg04836038, cg04846343, cg04847826, cg04859706, cg04875128, cg04885881, cg04902443, cg04907325, cg04980928, cg05021355, cg05024939, cg05067995, cg05169665, cg05185749, cg05187965, cg05196720, cg05201093, cg05230392, cg05236757, cg05239544, cg05258736, cg05270922, cg05280814, cg05298224, cg05331789, cg05338009, cg05352464, cg05380920, cg05399221, cg05418947, cg05422344, cg05423392, cg05426134, cg05442477, cg05481257, cg05485520, cg05498943, cg05506600, cg05517880, cg05551469, cg05575921, cg05616010, cg05635388, cg05666515, cg05669298, cg05671603, cg05676441, cg05679108, cg05715050, cg05720678, cg05721485, cg05726118, cg05729185, cg05777089, cg05786053, cg05802075, cg05820243, cg05828191, cg05830220, cg05845765, cg05901141, cg05910038, cg05913921, cg05941525, cg05951558, cg05987787, cg05989533, cg06001484, cg06030695, cg06047828, cg06062604, cg06068545, cg06072257, cg06083423, cg06159131, cg06169746, cg06213468, cg06232874, cg06251791, cg06279632, cg06295952, cg06319999, cg06335143, cg06374715, cg06401979, cg06429010, cg06454296, cg06478823, cg06519334, cg06535156, cg06572713, cg06572849, cg06580943, cg06602086, cg06604227, cg06610988, cg06621358, cg06630425, cg06639320, cg06642354, cg06645033, cg06660395, cg06665890, cg06667732, cg06672424, cg06675893, cg06679261, cg06683719, cg06692384, cg06696596, cg06753303, cg06784991, cg06789517, cg06812822, cg06870213, cg06874063, cg06878994, cg06888566, cg06926961, cg06958563, cg06975196, cg06984463, cg06997226, cg07015424, cg07023764, cg07057549, cg07076816, cg07097098, cg07151966, cg07193470, cg07196593, cg07202981, cg07304986, cg07323488, cg07351787, cg07363137, cg07365979, cg07393369, cg07404706, cg07426101, cg07434188, cg07462861, cg07529122, cg07541744, cg07558704, cg07569063, cg07601816, cg07613945, cg07620230, cg07624842, cg07629158, cg07640648, cg07643355, cg07668501, cg07672814, cg07674095, cg07675998, cg07692435, cg07702236, cg07714289, cg07768696, cg07778205, cg07828121, cg07855914, cg07871442, cg07871717, cg07899016, cg07951728, cg08075719, cg08094551, cg08103988, cg08113373, cg08125436, cg08126148, cg08132054, cg08173263, cg08182270, cg08259413, cg08281455, cg08284801, cg08312389, cg08335107, cg08342619, cg08350092, cg08369164, cg08375139, cg08421807, cg08424491, cg08439752, cg08443038, cg08506743, cg08551532, cg08573404, cg08609249, cg08640901, cg08684580, cg08709765, cg08743947, cg08750459, cg08762603, cg08772194, cg08812936, cg08830214, cg08856764, cg08885224, cg08893036, cg08968830, cg08992229, cg09082985, cg09087222, cg09087669, cg09094866, cg09103812, cg09129997, cg09146197, cg09167379, cg09169335, cg09191731, cg09196193, cg09211372, cg09228052, cg09267188, cg09313734, cg09317728, cg09365138, cg09391799, cg09422291, cg09428537, cg09434029, cg09435875, cg09450907, cg09463001, cg09507782, cg09536390, cg09554406, cg09576223, cg09601629, cg09621460, cg09681594, cg09702859, cg09703048, cg09716613, cg09717927, cg09717936, cg09788524, cg09823288, cg09823713, cg09831942, cg09842685, cg09862733, cg09868598, cg09874992, cg09931939, cg09963257, cg09969145, cg09977126, cg09993780, cg10043414, cg10060499, cg10116893, cg10123377, cg10127085, cg10133386, cg10157184, cg10168894, cg10201382, cg10208550, cg10235817, cg10251070, cg10290474, cg10325507, cg10326287, cg10407585, cg10459672, cg10472537, cg10501210, cg10514580, cg10518439, cg10573018, cg10583266, cg10601230, cg10621420, cg10624914, cg10662975, cg10703784, cg10716444, cg10759025, cg10766585, cg10859114, cg10866445, cg10874644, cg10878114, cg10882522, cg10884112, cg10949164, cg10961323, cg10977392, cg10996589, cg11020001, cg11031278, cg11052535, cg11104177, cg11113469, cg11120021, cg11130780, cg11149163, cg11240908, cg11241278, cg11270070, cg11290628, cg11338041, cg11341981, cg11362848, cg11400286, cg11408477, cg11409205, cg11415498, cg11426563, cg11479653, cg11532431, cg11540692, cg11620909, cg11624211, cg11640253, cg11649376, cg11671400, cg11691908, cg11725835, cg11735780, cg11737758, cg11749817, cg11752250, cg11765003, cg11777043, cg11793132, cg11801086, cg11804239, cg11819402, cg11824202, cg11833003, cg11844835, cg11921111, cg11935615, cg11951427, cg11971423, cg11976052, cg12016314, cg12016809, cg12018436, cg12030941, cg12038299, cg12050358, cg12100785, cg12112853, cg12135976, cg12145721, cg12146151, cg12151545, cg12158389, cg12159504, cg12168623, cg12170419, cg12195980, cg12242038, cg12272488, cg12282371, cg12307092, cg12347617, cg12364808, cg12378187, cg12394821, cg12416359, cg12434362, cg12463857, cg12518665, cg12538421, cg12560256, cg12569593, cg12586992, cg12606517, cg12615235, cg12637448, cg12655260, cg12662084, cg12743638, cg12744031, cg12797254, cg12853426, cg12935715, cg12941754, cg12978573, cg12986327, cg13017801, cg13055484, cg13073141, cg13083251, cg13087259, cg13099106, cg13154925, cg13167828, cg13169967, cg13176778, cg13188098, cg13206721, cg13224583, cg13229291, cg13234653, cg13257436, cg13295463, cg13298859, cg13312094, cg13340220, cg13343932, cg13367272, cg13369416, cg13372562, cg13393601, cg13399544, cg13431929, cg13473880, cg13507563, cg13543436, cg13545297, cg13563074, cg13609948, cg13671730, cg13672444, cg13676215, cg13690812, cg13697829, cg13702196, cg13714975, cg13719443, cg13742305, cg13779009, cg13814747, cg13897675, cg13914486, cg13941682, cg13959647, cg13979305, cg13987110, cg14030785, cg14047961, cg14117377, cg14118991, cg14157578, cg14167430, cg14172633, cg14175748, cg14209041, cg14224028, cg14234520, cg14251509, cg14252140, cg14257435, cg14262357, cg14264125, cg14280181, cg14339287, cg14354398, cg14384748, cg14391737, cg14413904, cg14441271, cg14442890, cg14484844, cg14534474, cg14615491, cg14639293, cg14640588, cg14675598, cg14688662, cg14708893, cg14710896, cg14738170, cg14751023, cg14781394, cg14794281, cg14801398, cg14837598, cg14850945, cg14873958, cg14920334, cg14942542, cg14962363, cg14975009, cg14994914, cg15033140, cg15047582, cg15059204, cg15109292, cg15128785, cg15141757, cg15143202, cg15175143, cg15179011, cg15194100, cg15208687, cg15223345, cg15234627, cg15246131, cg15288815, cg15318748, cg15374686, cg15391651, cg15405515, cg15418593, cg15436354, cg15467112, cg15471501, cg15480367, cg15498435, cg15506060, cg15575700, cg15592143, cg15603964, cg15625940, cg15690995, cg15709989, cg15710198, cg15720821, cg15733713, cg15821589, cg15827092, cg15837235, cg15885703, cg15909933, cg15961042, cg15968648, cg15994304, cg16014202, cg16014412, cg16026114, cg16070035, cg16084969, cg16097191, cg16141165, cg16195970, cg16198075, cg16211224, cg16240781, cg16306900, cg16314089, cg16369168, cg16408865, cg16430267, cg16488565, cg16489878, cg16517790, cg16529483, cg16551483, cg16569522, cg16570202, cg16649588, cg16661213, cg16679001, cg16733203, cg16736896, cg16769369, cg16795184, cg16842060, cg16861968, cg16882373, cg16891431, cg16908208, cg16969368, cg16979095, cg16987876, cg16994082, cg17017746, cg17030092, cg17050472, cg17077421, cg17107501, cg17117243, cg17120358, cg17129809, cg17133012, cg17148219, cg17210497, cg17219432, cg17225923, cg17232357, cg17251114, cg17275047, cg17408993, cg17435901, cg17464298, cg17464436, cg17465569, cg17476910, cg17485717, cg17514701, cg17569235, cg17576228, cg17591595, cg17652174, cg17660663, cg17665268, cg17691612, cg17695543, cg17699330, cg17761940, cg17786697, cg17825846, cg17872672, cg17885794, cg17944885, cg17945419, cg17967443, cg17979595, cg17981101, cg17988830, cg18014413, cg18023598, cg18031708, cg18031880, cg18071894, cg18133309, cg18181703, cg18190506, cg18251449, cg18262201, cg18319024, cg18380497, cg18398019, cg18425341, cg18516816, cg18587063, cg18597252, cg18598029, cg18604560, cg18669861, cg18699242, cg18740583, cg18771034, cg18784500, cg18795169, cg18817318, cg18822243, cg18856359, cg18859711, cg18882407, cg18885210, cg18976649, cg19006431, cg19009870, cg19058685, cg19089073, cg19091784, cg19172278, cg19182279, cg19224645, cg19225755, cg19266329, cg19284211, cg19296509, cg19297245, cg19302996, cg19304797, cg19308663, cg19320294, cg19380156, cg19474865, cg19536401, cg19548511, cg19558933, cg19604384, cg19611570, cg19635897, cg19636673, cg19652271, cg19665696, cg19693031, cg19726712, cg19730248, cg19774733, cg19800670, cg19822434, cg19859910, cg19909400, cg19935065, cg19941446, cg19977924, cg19992304, cg20004147, cg20025086, cg20037601, cg20059597, cg20119148, cg20128319, cg20143092, cg20155714, cg20162239, cg20185773, cg20192259, cg20207443, cg20217702, cg20244340, cg20249402, cg20270599, cg20272789, cg20282550, cg20299697, cg20348196, cg20351734, cg20353489, cg20370296, cg20376953, cg20432211, cg20442128, cg20446824, cg20515136, cg20534585, cg20587151, cg20594802, cg20596493, cg20597382, cg20597477, cg20601028, cg20623853, cg20628696, cg20656525, cg20663241, cg20668177, cg20675702, cg20720208, cg20737582, cg20773320, cg20802942, cg20865054, cg20874261, cg20891771, cg20907051, cg20914958, cg20918218, cg20921972, cg20972814, cg20973476, cg21000454, cg21077074, cg21082913, cg21112943, cg21123355, cg21124655, cg21132686, cg21136789, cg21185661, cg21204522, cg21232671, cg21253043, cg21259215, cg21305555, cg21306240, cg21312434, cg21314942, cg21321148, cg21346818, cg21356998, cg21410629, cg21447975, cg21455901, cg21489638, cg21533743, cg21566642, cg21622536, cg21628390, cg21640508, cg21658235, cg21693883, cg21717369, cg21723004, cg21725954, cg21730080, cg21733098, cg21739934, cg21761881, cg21783047, cg21791657, cg21824343, cg21834645, cg21870393, cg21907521, cg21934504, cg21947254, cg21962907, cg21985831, cg21986679, cg21998324, cg22002034, cg22019884, cg22022821, cg22023206, cg22041417, cg22061523, cg22061769, cg22069247, cg22171714, cg22178367, cg22200330, cg22220710, cg22232492, cg22243260, cg22282672, cg22322653, cg22339775, cg22344631, cg22356226, cg22363327, cg22416596, cg22434421, cg22442430, cg22454769, cg22556008, cg22563987, cg22581353, cg22594985, cg22691256, cg22711187, cg22719651, cg22729726, cg22736354, cg22792461, cg22822140, cg22822803, cg22945467, cg22956922, cg22993154, cg23059366, cg23063236, cg23080422, cg23192918, cg23196035, cg23234847, cg23235141, cg23248055, cg23265105, cg23276290, cg23302570, cg23313424, cg23366493, cg23371851, cg23378546, cg23410824, cg23424407, cg23447131, cg23465508, cg23486701, cg23500537, cg23547619, cg23549902, cg23561053, cg23570968, cg23574427, cg23586821, cg23615892, cg23643435, cg23793194, cg23838503, cg23879118, cg23883522, cg23894058, cg23910413, cg23923562, cg23956385, cg23964820, cg24003560, cg24027965, cg24055030, cg24055461, cg24112628, cg24141036, cg24185013, cg24220948, cg24236938, cg24241488, cg24262996, cg24266506, cg24293344, cg24317857, cg24337318, cg24354818, cg24362016, cg24365657, cg24372256, cg24392971, cg24397183, cg24432675, cg24463073, cg24468603, cg24492140, cg24507955, cg24520070, cg24523415, cg24530234, cg24543255, cg24577941, cg24589102, cg24601404, cg24674445, cg24678611, cg24697099, cg24721873, cg24769628, cg24796651, cg24799795, cg24835295, cg24920358, cg24925701, cg24967099, cg24976744, cg24982343, cg24997750, cg25047618, cg25063538, cg25114611, cg25146575, cg25148589, cg25156198, cg25161899, cg25181997, cg25189904, cg25202593, cg25259149, cg25259707, cg25330725, cg25331593, cg25361850, cg25375711, cg25397922, cg25418001, cg25418670, cg25420482, cg25420747, cg25436217, cg25499552, cg25504214, cg25560772, cg25563198, cg25569590, cg25623035, cg25638549, cg25639566, cg25670171, cg25671708, cg25703588, cg25717260, cg25738786, cg25745729, cg25784308, cg25786980, cg25843115, cg25843873, cg25903319, cg25916759, cg25927838, cg25928819, cg25943804, cg25945293, cg25952581, cg25952596, cg26029902, cg26044490, cg26071551, cg26093148, cg26116542, cg26116556, cg26156626, cg26229024, cg26239485, cg26267310, cg26276120, cg26301353, cg26314853, cg26315261, cg26441486, cg26442630, cg26443646, cg26479667, cg26499822, cg26500093, cg26501477, cg26528000, cg26544857, cg26577320, cg26599630, cg26606789, cg26676765, cg26691434, cg26702770, cg26760942, cg26764761, cg26767387, cg26805839, cg26818257, cg26839512, cg26883048, cg26962439, cg26992381, cg27021634, cg27066201, cg27079776, cg27127090, cg27128734, cg27130665, cg27131142, cg27152890, cg27154679, cg27154725, cg27208536, cg27229100, cg27245649, cg27256066, cg27296119, cg27315314, cg27336261, cg27348423, cg27354537, cg27357902, cg27368039, cg27374247, cg27395675, cg27449200, cg27454650, cg27461310, cg27488658, cg27498980, cg27500647, cg27553457, cg27589058, cg27632248.

The absence of methylation at any of the above identified CG loci correlates with and is predictive of one or more of the following diseases, disorders or conditions: alcohol liver disease, allergic rhinitis, aortic aneurysm dissection, arterial embolism thrombosis, asthma, bladder cancer, breast cancer, bronchiectasis, cancer, cardiomyopathy, chronic bronchitis, chronic kidney disease, chronic liver disease, chronic pulmonary heart disease, chronic viral hepatitis, cirrhosis, coin lesion lung disease, colon Rectum cancer, congestive heart failure, chronic obstructive pulmonary disease (COPD), coronary artery disease, cardiovascular disease (CVD) excluding stroke, depression, emphysema, hypertensive heart disease, interstitial lung disease, leukemia, liver cancer, lung cancer, melanoma, myocardial infraction, non-alcoholic fatty liver disease (NAFLD), non-Hodgkin lymphoma, other chronic hepatitis, ovarian cancer, pancreatic cancer, peripheral vascular disease, prostate cancer, stomach cancer, stroke, type 1 diabetes, type 2 diabetes, and uterine corpus cancer as verified by the genomic DNA collected from the reference group of individuals.

The number of CG loci observed from the indicated group of CG loci may vary from embodiment to embodiment of the method. Thus, the plurality of CG loci may be any number between 2 CG loci and 1,352 (all) of the CG loci of the indicated group. In one possible embodiment, a plurality of at least five CG loci are selected from the group. In another embodiment, at least ten CG loci are selected from the group. In another, at least 15 CG loci are selected from the group.

In yet another embodiment, a plurality of at least 25 CG loci are selected from the group. In another possible embodiment, a plurality of at least 50 CG loci are selected from the group. In another possible embodiment, a plurality of at least 75 CG loci are selected from the group. In another possible embodiment, a plurality of at least 100 CG loci are selected from the group. In another possible embodiment, a plurality of at least 150 CG loci are selected from the group.

In still another possible embodiment, a plurality of at least 200 CG loci are selected from the group. In another possible embodiment, a plurality of at least 250 CG loci are selected from the group. In another possible embodiment, a plurality of at least 500 CG loci are selected from the group. In another possible embodiment, a plurality of at least 1,000 CG loci are selected from the group. In yet another possible embodiment all 1,352 CG loci of the group are selected.

The bisulfite conversion process referenced in step (b) is known in the art. Kits useful for bisulfite conversion are commercially available from a number of manufacturers including Human Genetic Signatures' Methyleasy and Chemicon's CpGenome Modification Kit. See also, WO04096825A1, which describes bisulfite modification methods and Olek et al. Nuc. Acids Res. 24:5064-6 (1994), which discloses methods of performing bisulfite treatment and subsequent amplification.

The performing of the bisulfite conversion process may include the steps of amplifying the genomic DNA of the blood specimen by a polymerase chain reaction process, hybridizing the genomic DNA to a beadchip to create a genomic DNA hybridized beadchip, staining the genomic DNA hybridized beadchip and then imaging the genomic DNA hybridized beadchip.

The imaging of the genomic DNA hybridized beadchip may be performed using the Illumina EPIC850k array which provides for unambiguous CG loci identification for purposes of observing methylation at specific CG loci of the human DNA genome. Alternatively, other methods of epigenetic beta value collection may be used if desired.

The method also includes the step of using a regression algorithm to correlate the unmethylated CG loci observed in (b) with the unmethylated GC loci observed in the reference group of human individuals. This includes performing, by computing device, a penalized regression model to create the regression algorithm of CpGs with relative weights wherein a methylation risk score (MRS) is defined by linear combination of m CpG site beta values c and weights w where: MRS=Σi=1mwici. It should be appreciated that different relative weights may be assigned to the GC loci.

To ensure the methylation risk score added predictive value over commonly captured features (e.g., age and sex), we created a baseline predictive model that included patients' age, sex, reference-based methylation cell-type composition estimates, self-reported race-ethnicity, self-reported smoking status, and the first ten genetic principal components. We fit the baseline model using a linear or logistic regression model depending on whether the outcome was continuous or binary. We compared the baseline model to models that included the baseline features as well as cither methylation or genotype data. For the MRS, we used regression with LASSO, elastic net, and ridge regularization over the genomic features while treating the baseline features as fixed covariates. We fit all models using 10-fold double cross-validation, wherein each training set an additional cross-validation was performed for hyperparameter selection, then this training-set cross-validated model was used to predict the held-out test set. We tested for significance using an association test (via linear regression) between the cross-validated predicted outcome (i.e., the concatenated predictor across all folds) and the true outcome.

Still further, the method includes predicting proteomic and metabolomic data as surrogate biomarkers. For instance, we are using DNA methylation to predict things like C-Reactive protein which is a classical clinical marker. Toward this end, a plurality of known proteins and metabolites of medical interest for diagnosing alcohol liver disease, allergic rhinitis, aortic aneurysm dissection, arterial embolism thrombosis, asthma, bladder cancer, breast cancer, bronchiectasis, cancer, cardiomyopathy, chronic bronchitis, chronic kidney disease, chronic liver disease, chronic pulmonary heart disease, chronic viral hepatitis, cirrhosis, coin lesion lung disease, colon Rectum cancer, congestive heart failure, chronic obstructive pulmonary disease (COPD), coronary artery disease, cardiovascular disease (CVD) excluding stroke, depression, emphysema, hypertensive heart disease, interstitial lung disease, leukemia, liver cancer, lung cancer, melanoma, myocardial infraction, non-alcoholic fatty liver disease (NAFLD), non-Hodgkin lymphoma, other chronic hepatitis, ovarian cancer, pancreatic cancer, peripheral vascular disease, prostate cancer, stomach cancer, stroke, type 1 diabetes, type 2 diabetes, and uterine corpus cancer are initially used to construct a baseline CG loci reference matrix from a reference group of individuals with known health histories. The method is then used to predict the levels of those plurality of proteins and metabolites in the human subject by correlating the unmethylated CG loci observed in (b) with the unmethylated CG loci observed in the reference group of human individuals to predict disease risk or health of the human subject.

Each of the following terms written in singular grammatical form: “a”, “an”, and “the”, as used herein, means “at least one”, or “one or more”. Use of the phrase “One or more” herein does not alter this intended meaning of “a”, “an”, or “the”. Accordingly, the terms “a”, “an”, and “the”, as used herein, may also refer to, and encompass, a plurality of the stated entity or object, unless otherwise specifically defined or stated herein, or, unless the context clearly dictates otherwise. For example, the phrase: “a protein”, as used herein, may also refer to, and encompass, a plurality of proteins.

Each of the following terms: “includes”, “including”, “has”, “having”, “comprises”, and “comprising”, and, their linguistic/grammatical variants, derivatives, or/and conjugates, as used herein, means “including, but not limited to”, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof.

The phrase “consisting of”, as used herein, is closed-ended and excludes any element, step, or ingredient not specifically mentioned. The phrase “consisting essentially of”, as used herein, is a semi-closed term indicating that an item is limited to the components specified and those that do not materially affect the basic and novel characteristic(s) of what is specified.

Terms of approximation, such as the terms about, substantially, approximately, etc., as used herein, refers to ±10% of the stated numerical value.

Although the method of this disclosure have been illustratively described and presented by way of specific exemplary embodiments, and examples thereof, it is evident that many alternatives, modifications, or/and variations, thereof, will be apparent to those skilled in the art. Accordingly, it is intended that all such alternatives, modifications, or/and variations, fall within the spirit of, and are encompassed by, the broad scope of the appended claims.

What is claimed:

1. A method, comprising:
(a) obtaining genomic DNA from a blood or saliva specimen of a human subject;
(b) observing unmethylated CG loci, in the genomic DNA of the human subject, wherein said observing includes performing a bisulfite conversion process on the genomic DNA of the human subject so that cytosine residues in the genomic DNA of the human subject are transformed to uracil, while 5-methylcytosine residues in the genomic DNA of the human subject are not transformed to uracil;
(c) comparing the unmethylated CG loci observed in (b) to unmethylated CG loci observed in genomic DNA collected from a reference group of human individuals; and
(d) correlating the unmethylated CG loci observed in (b) with the unmethylated CG loci observed in the reference group of human individuals to predict health of the human subject wherein the CG loci are a plurality of at least five CG loci selected from a group consisting of cg00011943, cg00014020, cg00056433, cg00067133, cg00071360, cg00074145, cg00080105, cg00120123, cg00122614, cg00153759, cg00177243, cg00178119, cg00182994, cg00209129, cg00215550, cg00217225, cg00236988, cg00237714, cg00243480, cg00251125, cg00262132, cg00275686, cg00278547, cg00283022, cg00303773, cg00322993, cg00342468, cg00358895, cg00458337, cg00465312, cg00481382, cg00530438, cg00538458, cg00548856, cg00593298, cg00594167, cg00602326, cg00615271, cg00631329, cg00639633, cg00646555, cg00687674, cg00687962, cg00698355, cg00712841, cg00737155, cg00748449, cg00756943, cg00767058, cg00800095, cg00815399, cg00910115, cg00935026, cg00935421, cg00964035, cg00973876, cg00993310, cg01020263, cg01025883, cg01067216, cg01089474, cg01094440, cg01098936, cg01113710, cg01134012, cg01166985, cg01169463, cg01178063, cg01224520, cg01243823, cg01253289, cg01266159, cg01271129, cg01274656, cg01282852, cg01313513, cg01320510, cg01346718, cg01377932, cg01380346, cg01402735, cg01410359, cg01446515, cg01457077, cg01541424, cg01545493, cg01549940, cg01579765, cg01586635, cg01606057, cg01610915, cg01645869, cg01652244, cg01660630, cg01715525, cg01770232, cg01809675, cg01824466, cg01826979, cg01844642, cg01849284, cg01857379, cg01858517, cg01865937, cg01892689, cg01893681, cg01894498, cg01929138, cg01930417, cg01956293, cg02001279, cg02007867, cg02042600, cg02048674, cg02055264, cg02056921, cg02058108, cg02081263, cg02084118, cg02084211, cg02094681, cg02106682, cg02132714, cg02217269, cg02246605, cg02310386, cg02339455, cg02345572, cg02371935, cg02380750, cg02390624, cg02415029, cg02426324, cg02444928, cg02447542, cg02471378, cg02474731, cg02489377, cg02500990, cg02509300, cg02524236, cg02534780, cg02605776, cg02613370, cg02618485, cg02643782, cg02672830, cg02675560, cg02720346, cg02721394, cg02731293, cg02740457, cg02762363, cg02768694, cg02798801, cg02867402, cg02909068, cg02942382, cg02981003, cg02987388, cg03007462, cg03031520, cg03044367, cg03082247, cg03137472, cg03138091, cg03139710, cg03240800, cg03245912, cg03261737, cg03328727, cg03340356, cg03340754, cg03356492, cg03357219, cg03368099, cg03400443, cg03401077, cg03417454, cg03423767, cg03441242, cg03443888, cg03458265, cg03463465, cg03474430, cg03514545, cg03517760, cg03546163, cg03549506, cg03549779, cg03550384, cg03558379, cg03587144, cg03593336, cg03605735, cg03626541, cg03636183, cg03644691, cg03650179, cg03654623, cg03676636, cg03707183, cg03732728, cg03741185, cg03741498, cg03743982, cg03760060, cg03772253, cg03785619, cg03796580, cg03807316, cg03834947, cg03839794, cg03858365, cg03874914, cg03876766, cg03877420, cg03926921, cg03935495, cg03945604, cg03989967, cg04030486, cg04045009, cg04046848, cg04060356, cg04064054, cg04079779, cg04083076, cg04114590, cg04115418, cg04137003, cg04139465, cg04148839, cg04156293, cg04161236, cg04162034, cg04172953, cg04181107, cg04204356, cg04206342, cg04213565, cg04214966, cg04225172, cg04227949, cg04230438, cg04235071, cg04241623, cg04276953, cg04285652, cg04322572, cg04393397, cg04398451, cg04411086, cg04436120, cg04439218, cg04444415, cg04460372, cg04480106, cg04484376, cg04545003, cg04567124, cg04584675, cg04595658, cg04615668, cg04707706, cg04733838, cg04742282, cg04757168, cg04768425, cg04781339, cg04789125, cg04799410, cg04836038, cg04846343, cg04847826, cg04859706, cg04875128, cg04885881, cg04902443, cg04907325, cg04980928, cg05021355, cg05024939, cg05067995, cg05169665, cg05185749, cg05187965, cg05196720, cg05201093, cg05230392, cg05236757, cg05239544, cg05258736, cg05270922, cg05280814, cg05298224, cg05331789, cg05338009, cg05352464, cg05380920, cg05399221, cg05418947, cg05422344, cg05423392, cg05426134, cg05442477, cg05481257, cg05485520, cg05498943, cg05506600, cg05517880, cg05551469, cg05575921, cg05616010, cg05635388, cg05666515, cg05669298, cg05671603, cg05676441, cg05679108, cg05715050, cg05720678, cg05721485, cg05726118, cg05729185, cg05777089, cg05786053, cg05802075, cg05820243, cg05828191, cg05830220, cg05845765, cg05901141, cg05910038, cg05913921, cg05941525, cg05951558, cg05987787, cg05989533, cg06001484, cg06030695, cg06047828, cg06062604, cg06068545, cg06072257, cg06083423, cg06159131, cg06169746, cg06213468, cg06232874, cg06251791, cg06279632, cg06295952, cg06319999, cg06335143, cg06374715, cg06401979, cg06429010, cg06454296, cg06478823, cg06519334, cg06535156, cg06572713, cg06572849, cg06580943, cg06602086, cg06604227, cg06610988, cg06621358, cg06630425, cg06639320, cg06642354, cg06645033, cg06660395, cg06665890, cg06667732, cg06672424, cg06675893, cg06679261, cg06683719, cg06692384, cg06696596, cg06753303, cg06784991, cg06789517, cg06812822, cg06870213, cg06874063, cg06878994, cg06888566, cg06926961, cg06958563, cg06975196, cg06984463, cg06997226, cg07015424, cg07023764, cg07057549, cg07076816, cg07097098, cg07151966, cg07193470, cg07196593, cg07202981, cg07304986, cg07323488, cg07351787, cg07363137, cg07365979, cg07393369, cg07404706, cg07426101, cg07434188, cg07462861, cg07529122, cg07541744, cg07558704, cg07569063, cg07601816, cg07613945, cg07620230, cg07624842, cg07629158, cg07640648, cg07643355, cg07668501, cg07672814, cg07674095, cg07675998, cg07692435, cg07702236, cg07714289, cg07768696, cg07778205, cg07828121, cg07855914, cg07871442, cg07871717, cg07899016, cg07951728, cg08075719, cg08094551, cg08103988, cg08113373, cg08125436, cg08126148, cg08132054, cg08173263, cg08182270, cg08259413, cg08281455, cg08284801, cg08312389, cg08335107, cg08342619, cg08350092, cg08369164, cg08375139, cg08421807, cg08424491, cg08439752, cg08443038, cg08506743, cg08551532, cg08573404, cg08609249, cg08640901, cg08684580, cg08709765, cg08743947, cg08750459, cg08762603, cg08772194, cg08812936, cg08830214, cg08856764, cg08885224, cg08893036, cg08968830, cg08992229, cg09082985, cg09087222, cg09087669, cg09094866, cg09103812, cg09129997, cg09146197, cg09167379, cg09169335, cg09191731, cg09196193, cg09211372, cg09228052, cg09267188, cg09313734, cg09317728, cg09365138, cg09391799, cg09422291, cg09428537, cg09434029, cg09435875, cg09450907, cg09463001, cg09507782, cg09536390, cg09554406, cg09576223, cg09601629, cg09621460, cg09681594, cg09702859, cg09703048, cg09716613, cg09717927, cg09717936, cg09788524, cg09823288, cg09823713, cg09831942, cg09842685, cg09862733, cg09868598, cg09874992, cg09931939, cg09963257, cg09969145, cg09977126, cg09993780, cg10043414, cg10060499, cg10116893, cg10123377, cg10127085, cg10133386, cg10157184, cg10168894, cg10201382, cg10208550, cg10235817, cg10251070, cg10290474, cg10325507, cg10326287, cg10407585, cg10459672, cg10472537, cg10501210, cg10514580, cg10518439, cg10573018, cg10583266, cg10601230, cg10621420, cg10624914, cg10662975, cg10703784, cg10716444, cg10759025, cg10766585, cg10859114, cg10866445, cg10874644, cg10878114, cg10882522, cg10884112, cg10949164, cg10961323, cg10977392, cg10996589, cg11020001, cg11031278, cg11052535, cg11104177, cg11113469, cg11120021, cg11130780, cg11149163, cg11240908, cg11241278, cg11270070, cg11290628, cg11338041, cg11341981, cg11362848, cg11400286, cg11408477, cg11409205, cg11415498, cg11426563, cg11479653, cg11532431, cg11540692, cg11620909, cg11624211, cg11640253, cg11649376, cg11671400, cg11691908, cg11725835, cg11735780, cg11737758, cg11749817, cg11752250, cg11765003, cg11777043, cg11793132, cg11801086, cg11804239, cg11819402, cg11824202, cg11833003, cg11844835, cg11921111, cg11935615, cg11951427, cg11971423, cg11976052, cg12016314, cg12016809, cg12018436, cg12030941, cg12038299, cg12050358, cg12100785, cg12112853, cg12135976, cg12145721, cg12146151, cg12151545, cg12158389, cg12159504, cg12168623, cg12170419, cg12195980, cg12242038, cg12272488, cg12282371, cg12307092, cg12347617, cg12364808, cg12378187, cg12394821, cg12416359, cg12434362, cg12463857, cg12518665, cg12538421, cg12560256, cg12569593, cg12586992, cg12606517, cg12615235, cg12637448, cg12655260, cg12662084, cg12743638, cg12744031, cg12797254, cg12853426, cg12935715, cg12941754, cg12978573, cg12986327, cg13017801, cg13055484, cg13073141, cg13083251, cg13087259, cg13099106, cg13154925, cg13167828, cg13169967, cg13176778, cg13188098, cg13206721, cg13224583, cg13229291, cg13234653, cg13257436, cg13295463, cg13298859, cg13312094, cg13340220, cg13343932, cg13367272, cg13369416, cg13372562, cg13393601, cg13399544, cg13431929, cg13473880, cg13507563, cg13543436, cg13545297, cg13563074, cg13609948, cg13671730, cg13672444, cg13676215, cg13690812, cg13697829, cg13702196, cg13714975, cg13719443, cg13742305, cg13779009, cg13814747, cg13897675, cg13914486, cg13941682, cg13959647, cg13979305, cg13987110, cg14030785, cg14047961, cg14117377, cg14118991, cg14157578, cg14167430, cg14172633, cg14175748, cg14209041, cg14224028, cg14234520, cg14251509, cg14252140, cg14257435, cg14262357, cg14264125, cg14280181, cg14339287, cg14354398, cg14384748, cg14391737, cg14413904, cg14441271, cg14442890, cg14484844, cg14534474, cg14615491, cg14639293, cg14640588, cg14675598, cg14688662, cg14708893, cg14710896, cg14738170, cg14751023, cg14781394, cg14794281, cg14801398, cg14837598, cg14850945, cg14873958, cg14920334, cg14942542, cg14962363, cg14975009, cg14994914, cg15033140, cg15047582, cg15059204, cg15109292, cg15128785, cg15141757, cg15143202, cg15175143, cg15179011, cg15194100, cg15208687, cg15223345, cg15234627, cg15246131, cg15288815, cg15318748, cg15374686, cg15391651, cg15405515, cg15418593, cg15436354, cg15467112, cg15471501, cg15480367, cg15498435, cg15506060, cg15575700, cg15592143, cg15603964, cg15625940, cg15690995, cg15709989, cg15710198, cg15720821, cg15733713, cg15821589, cg15827092, cg15837235, cg15885703, cg15909933, cg15961042, cg15968648, cg15994304, cg16014202, cg16014412, cg16026114, cg16070035, cg16084969, cg16097191, cg16141165, cg16195970, cg16198075, cg16211224, cg16240781, cg16306900, cg16314089, cg16369168, cg16408865, cg16430267, cg16488565, cg16489878, cg16517790, cg16529483, cg16551483, cg16569522, cg16570202, cg16649588, cg16661213, cg16679001, cg16733203, cg16736896, cg16769369, cg16795184, cg16842060, cg16861968, cg16882373, cg16891431, cg16908208, cg16969368, cg16979095, cg16987876, cg16994082, cg17017746, cg17030092, cg17050472, cg17077421, cg17107501, cg17117243, cg17120358, cg17129809, cg17133012, cg17148219, cg17210497, cg17219432, cg17225923, cg17232357, cg17251114, cg17275047, cg17408993, cg17435901, cg17464298, cg17464436, cg17465569, cg17476910, cg17485717, cg17514701, cg17569235, cg17576228, cg17591595, cg17652174, cg17660663, cg17665268, cg17691612, cg17695543, cg17699330, cg17761940, cg17786697, cg17825846, cg17872672, cg17885794, cg17944885, cg17945419, cg17967443, cg17979595, cg17981101, cg17988830, cg18014413, cg18023598, cg18031708, cg18031880, cg18071894, cg18133309, cg18181703, cg18190506, cg18251449, cg18262201, cg18319024, cg18380497, cg18398019, cg18425341, cg18516816, cg18587063, cg18597252, cg18598029, cg18604560, cg18669861, cg18699242, cg18740583, cg18771034, cg18784500, cg18795169, cg18817318, cg18822243, cg18856359, cg18859711, cg18882407, cg18885210, cg18976649, cg19006431, cg19009870, cg19058685, cg19089073, cg19091784, cg19172278, cg19182279, cg19224645, cg19225755, cg19266329, cg19284211, cg19296509, cg19297245, cg19302996, cg19304797, cg19308663, cg19320294, cg19380156, cg19474865, cg19536401, cg19548511, cg19558933, cg19604384, cg19611570, cg19635897, cg19636673, cg19652271, cg19665696, cg19693031, cg19726712, cg19730248, cg19774733, cg19800670, cg19822434, cg19859910, cg19909400, cg19935065, cg19941446, cg19977924, cg19992304, cg20004147, cg20025086, cg20037601, cg20059597, cg20119148, cg20128319, cg20143092, cg20155714, cg20162239, cg20185773, cg20192259, cg20207443, cg20217702, cg20244340, cg20249402, cg20270599, cg20272789, cg20282550, cg20299697, cg20348196, cg20351734, cg20353489, cg20370296, cg20376953, cg20432211, cg20442128, cg20446824, cg20515136, cg20534585, cg20587151, cg20594802, cg20596493, cg20597382, cg20597477, cg20601028, cg20623853, cg20628696, cg20656525, cg20663241, cg20668177, cg20675702, cg20720208, cg20737582, cg20773320, cg20802942, cg20865054, cg20874261, cg20891771, cg20907051, cg20914958, cg20918218, cg20921972, cg20972814, cg20973476, cg21000454, cg21077074, cg21082913, cg21112943, cg21123355, cg21124655, cg21132686, cg21136789, cg21185661, cg21204522, cg21232671, cg21253043, cg21259215, cg21305555, cg21306240, cg21312434, cg21314942, cg21321148, cg21346818, cg21356998, cg21410629, cg21447975, cg21455901, cg21489638, cg21533743, cg21566642, cg21622536, cg21628390, cg21640508, cg21658235, cg21693883, cg21717369, cg21723004, cg21725954, cg21730080, cg21733098, cg21739934, cg21761881, cg21783047, cg21791657, cg21824343, cg21834645, cg21870393, cg21907521, cg21934504, cg21947254, cg21962907, cg21985831, cg21986679, cg21998324, cg22002034, cg22019884, cg22022821, cg22023206, cg22041417, cg22061523, cg22061769, cg22069247, cg22171714, cg22178367, cg22200330, cg22220710, cg22232492, cg22243260, cg22282672, cg22322653, cg22339775, cg22344631, cg22356226, cg22363327, cg22416596, cg22434421, cg22442430, cg22454769, cg22556008, cg22563987, cg22581353, cg22594985, cg22691256, cg22711187, cg22719651, cg22729726, cg22736354, cg22792461, cg22822140, cg22822803, cg22945467, cg22956922, cg22993154, cg23059366, cg23063236, cg23080422, cg23192918, cg23196035, cg23234847, cg23235141, cg23248055, cg23265105, cg23276290, cg23302570, cg23313424, cg23366493, cg23371851, cg23378546, cg23410824, cg23424407, cg23447131, cg23465508, cg23486701, cg23500537, cg23547619, cg23549902, cg23561053, cg23570968, cg23574427, cg23586821, cg23615892, cg23643435, cg23793194, cg23838503, cg23879118, cg23883522, cg23894058, cg23910413, cg23923562, cg23956385, cg23964820, cg24003560, cg24027965, cg24055030, cg24055461, cg24112628, cg24141036, cg24185013, cg24220948, cg24236938, cg24241488, cg24262996, cg24266506, cg24293344, cg24317857, cg24337318, cg24354818, cg24362016, cg24365657, cg24372256, cg24392971, cg24397183, cg24432675, cg24463073, cg24468603, cg24492140, cg24507955, cg24520070, cg24523415, cg24530234, cg24543255, cg24577941, cg24589102, cg24601404, cg24674445, cg24678611, cg24697099, cg24721873, cg24769628, cg24796651, cg24799795, cg24835295, cg24920358, cg24925701, cg24967099, cg24976744, cg24982343, cg24997750, cg25047618, cg25063538, cg25114611, cg25146575, cg25148589, cg25156198, cg25161899, cg25181997, cg25189904, cg25202593, cg25259149, cg25259707, cg25330725, cg25331593, cg25361850, cg25375711, cg25397922, cg25418001, cg25418670, cg25420482, cg25420747, cg25436217, cg25499552, cg25504214, cg25560772, cg25563198, cg25569590, cg25623035, cg25638549, cg25639566, cg25670171, cg25671708, cg25703588, cg25717260, cg25738786, cg25745729, cg25784308, cg25786980, cg25843115, cg25843873, cg25903319, cg25916759, cg25927838, cg25928819, cg25943804, cg25945293, cg25952581, cg25952596, cg26029902, cg26044490, cg26071551, cg26093148, cg26116542, cg26116556, cg26156626, cg26229024, cg26239485, cg26267310, cg26276120, cg26301353, cg26314853, cg26315261, cg26441486, cg26442630, cg26443646, cg26479667, cg26499822, cg26500093, cg26501477, cg26528000, cg26544857, cg26577320, cg26599630, cg26606789, cg26676765, cg26691434, cg26702770, cg26760942, cg26764761, cg26767387, cg26805839, cg26818257, cg26839512, cg26883048, cg26962439, cg26992381, cg27021634, cg27066201, cg27079776, cg27127090, cg27128734, cg27130665, cg27131142, cg27152890, cg27154679, cg27154725, cg27208536, cg27229100, cg27245649, cg27256066, cg27296119, cg27315314, cg27336261, cg27348423, cg27354537, cg27357902, cg27368039, cg27374247, cg27395675, cg27449200, cg27454650, cg27461310, cg27488658, cg27498980, cg27500647, cg27553457, cg27589058, cg27632248.

2. The method of claim 1, wherein a plurality of at least ten CG loci are selected from the group.

3. The method of claim 1, wherein a plurality of at least 15 CG loci are selected from the group.

4. The method of claim 1, wherein a plurality of at least 25 CG loci are selected from the group.

5. The method of claim 1, wherein a plurality of at least 50 CG loci are selected from the group.

6. The method of claim 1, wherein a plurality of at least 75 CG loci are selected from the group.

7. The method of claim 1, wherein a plurality of at least 100 CG loci are selected from the group.

8. The method of claim 1, wherein a plurality of at least 150 CG loci are selected from the group.

9. The method of claim 1, wherein a plurality of at least 200 CG loci are selected from the group.

10. The method of claim 1, wherein a plurality of at least 250 CG loci are selected from the group.

11. The method of claim 1, wherein a plurality of at least 500 CG loci are selected from the group.

12. The method of claim 1, wherein a plurality of at least 1000 CG loci are selected from the group.

13. The method of claim 1, wherein all 1,352 CG loci are selected from the group.

14. The method of claim 1, including amplifying the genomic DNA of the blood or saliva specimen by a polymerase chain reaction.

15. The method of claim 14, wherein the observing of the unmethylated CG loci, in the genomic DNA of the human subject includes hybridizing the genomic DNA to a beadchip to create a genomic DNA hybridized beadchip and then imaging the genomic DNA hybridized beadchip.

16. The method of claim 15, including using a regression algorithm to correlate the unmethylated CG loci observed in (b) with the unmethylated GC loci observed in the reference group of human individuals.

17. The method of claim 16, including performing, by computing device, a penalized regression model to create the regression algorithm of CpGs with relative weights wherein a methylation risk score (MRS) is defined by linear combination of m CpG site beta values c and weights w where: MRS=Σi=1mwici.

18. The method of claim 1, wherein the observing of the unmethylated CG loci, in the genomic DNA of the human subject includes hybridizing the genomic DNA to a beadchip to create a genomic DNA hybridized bead chip and then imaging the genomic DNA hybridized beadchip.
